(19) United States
(12) Patent Application Publication
(10) Pub. No.: US 2017/0121646 A1
(43) Pub. Date: May 4, 2017

(54) Improved Stabilization of Non-Protease Enzyme

(71) Applicant: Novozymes A/S, Bagsvaerd (DK)

(72) Inventor: Lise Munch Mikkelsen, Roedovre (DK)

(73) Assignee: Novozymes A/S, Bagsvaerd (DK)

(21) Appl. No.: 15/318,770

(22) PCT Filed: Jul. 1, 2015
(86) PCT No.: PCT/EP2015/065021

§ 371 Date: Dec. 14, 2016

(30) Foreign Application Priority Data
Jul. 3, 2014 (EP) 14175674.2

(51) Int. Cl. C11D 3/386; C12N 9/96

(52) U.S. Cl. CPC C11D 3/3869; C12N 9/96; C11D 3/38618; C11D 3/38627; C11D 3/38645; C11D 3/38663

(57) ABSTRACT

The stabilization of non-subtilisin enzymes by certain subtilin inhibitors in liquid detergents is particularly effective when the concentration of the subtilisin inhibitor is higher than a concentration C.

### REFERENCE TO A SEQUENCE LISTING

This application contains a Sequence Listing in computer readable form. The computer readable form is incorporated herein by reference.

### FIELD OF THE INVENTION

The present invention relates to stabilization of non-subtilisin enzymes in liquid detergent compositions.

### BACKGROUND

WO 98/13458, WO 94/04651, WO 98/13460, WO 95/25791, and WO 2009/118375 disclose liquid detergents with a subtilisin-type protease stabilized by a peptide aldehyde. WO 2011/036153 discloses that the addition of a peptide aldehyde to a particulate subtilisin-containing detergent can improve the detergency.

It is well known that aldehydes can form soluble adducts with NaHSO₃ (bisulfite or hydrosulfite adducts) and that peptide aldehydes tend to be sparingly water soluble. Peptide aldehyde hydrosulfite adducts, and their use in detergents, are disclosed in WO 2013/004636.

WO 98/47523 and U.S. Pat. No. 6,500,802 disclose peptidyl-2-amino-1-hydroxyalkanesulfonic acids and their use as protease inhibitors. U.S. Pat. No. 5,436,229 discloses bisulfite adducts of L-Arginine aldehyde derivatives and their use as thrombin inhibitors.

U.S. Pat. No. 4,703,036, U.S. Pat. No. 4,478,745 and U.S. Pat. No. 5,578,574 disclose methods of preparing peptide aldehydes in dry form.

### SUMMARY

The inventors have found that incorporation of a subtilisin inhibitor above a certain concentration into a liquid detergent improves the storage stability of non-subtilisin enzymes more than the storage stability of subtilisins.

Accordingly, the invention provides a method for improving the storage stability of a non-subtilisin enzyme in a liquid detergent composition containing a subtilisin and a non-subtilisin, comprising preparing a liquid detergent composition containing a subtilisin, a non-subtilisin, and more than a concentration C of a subtilisin inhibitor; wherein C is determined as the concentration of inhibitor where the relative residual activity of the subtilisin is increased by 25% of the increase from no inhibitor to C, when the concentration of the subtilisin inhibitor is increased from C to two times C, after incubation for one week at 40° C. in Detergent M.

In an embodiment, the relative storage stability improvement of the non-subtilisin is higher than the relative storage stability improvement of the subtilisin.

Other aspects and embodiments of the invention are apparent from the description and examples.

### DETAILED DESCRIPTION

We have found that the level of protease inhibitors needed for improving the stability of non-protease enzymes (like amylase, lipase, cellulase, and other detergent enzymes) is higher than for securing the stability of protease enzymes; and the stabilizing effect of a second addition of inhibitor on top of a first addition of inhibitor is higher on non-protease enzymes than on proteases. Hence by adding protease inhibitor to the individual non-protease enzyme products will lead to a more optimal inhibitor dosing for improving stability of the whole enzyme composition, and is easier and more convenient for a detergent manufacturer, than having to choose proteases with different inhibitor levels depending on the choice and inclusion rate of non-protease enzymes. Moreover the inhibitor doses will be “automatically adjusted” to the desired level.

The present invention relates to a detergent composition comprising a subtilisin, a non-subtilisin and a subtilisin inhibitor; in which the subtilisin inhibitor concentrations are above a certain concentration C. The first inhibitor addition (concentration C) primarily increases protease stability, whereas when the inhibitor concentration is above C, additional inhibitor addition will have a more pronounced effect on the stability of the non-protease enzyme than on the protease enzyme; and hence beneficially could be added together with the non-protease enzyme, as part of a non-protease product. The present invention thus also relates to a liquid composition comprising a non-protease enzyme and a protease inhibitor, and the method of using this for increasing stability of a multi enzyme solution in a protease containing liquid detergent, which may or may not already contain protease inhibitors.

### DEFINITIONS

### Inhibitor Concentration C

An inhibitor concentration C is defined, so that when the inhibitor concentration is doubled from a concentration of C to a concentration of 2C (i.e., 2C=2×C), the residual protease activity increases (“residual protease activity at 2C”−“residual protease activity at C”) by 25% of the residual protease activity increase from no inhibitor to C (“residual protease activity at C”−“residual activity at no inhibitor”). “No inhibitor” means that the concentration of inhibitor is zero.

The test system to determine “dose 1” (C) and “dose 2” (2C) is described in Example 1. A model detergent system is used (detergent A) and protease, lipase, and different concentrations of protease inhibitor are added (detergent M). Residual protease activity is determined by comparing activity after storage for 1 week at 40° C. with 1 week at −18° C. The data are used to prepare a dose response curve which is used to identify the inhibitor concentration C. Inhibitor concentrations are given in molar ratios and mg/L relative to the test system in Example 1 and mg/L will be similarly increased if the protease concentration is increased.

Compositions of the present invention can thus be described as detergent compositions with protease and non-protease enzymes having more than “dose 1” protease inhibitor (C); where

$$\frac{( \begin{matrix}{}^{``} residual protease activity at dose {2}^{″} - \\ {}^{``} residual protease activity at dose {1}^{″}\end{matrix} )}{( \begin{matrix}{}^{``} residual protease activity at dose {1}^{″} - \\ {}^{``} residual protease activity at no {inhibitor}^{″}\end{matrix} )} = 0.25 ;$$

and “dose 2”=2×“dose 1”.

### Subtilisin

The subtilisin enzymes for use in the present invention include those of bacterial, fungal, plant, viral or animal origin e.g. vegetable or microbial origin. Microbial origin is preferred. Chemically modified or protein engineered mutants are included.

In the context of the present invention, the subtilisin enzyme family (EC 3.4.21.62) shall be understood as described by Siezen et al., Protein Engng. 4 (1991) 719-737 and Siezen et al. Protein Science 6 (1997) 501-523. As described therein, the subtilisin family may be divided into 3 sub-groups, i.e. I-S1 (“true” subtilisins), I-S2 (highly alkaline proteases) and intracellular subtilisins.

Examples of subtilisins are those derived from Bacillus such as subtilisin lentus, Bacillus lentus, subtilisin Novo, subtilisin Carlsberg, Bacillus licheniformis, subtilisin BPN′, subtilisin 309, subtilisin 147 and subtilisin 168 described in WO 89/06279 and protease PD138 (WO 93/18140). Additional examples are described in WO 98/020115, WO 01/44452, WO 01/58275, WO 01/58276, WO 03/006602 and WO 04/099401.

Examples of useful variants are described in WO 92/19729, WO 98/20115, WO 98/20116, and WO 98/34946, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V4I, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V2051, L217D, N218D, M222S, A232V, K235L, Q236H, Q245R, N252K, T274A (using BPN′ numbering).

Examples of commercially available subtilisins include Kannase™, Everlase™, Primase™, Duralase™, Esperase™, Alcalase™, Durazym™, Savinase™, Ovozyme™, Liquanase™, Coronase™, Polarzyme™, Pyrase™, and Clear-Lens™ Pro; Blaze™ (Novozymes NS). Other commercially available proteases include Ronozyme™ Pro, Maxatase™Maxacal™, Maxapem™, Opticlean™, Properase™, Purafect™, Purafect Ox™, Purafact Prime™, Excellase™, FN2™, FN3™ and FN4™ (available from Dupont).

In an embodiment of the invention, the subtilisin is subtilisin 309 or subtilisin BPN′, or a variant of any of these. Preferably, the amino acid sequence of the subtilisin has at least 60% sequence identity, preferably at least 70%, more preferably at least 80%, more preferably at least 90%, more preferably at least 95%, 96%, 97%, 98%, 99%, and most preferably 100% sequence identity to the amino acid sequence of SEQ ID NO: 1 or SEQ ID NO: 2.

In an embodiment, the number of amino acid substitutions, deletions and/or insertions introduced into the amino acid sequence of SEQ ID NO: 1 or SEQ ID NO: 2 is up to 10, e.g., 1, 2, 3, 4, 5, 6, 7, 8, 9, or 10; or up to 5, e.g., 1, 2, 3, 4, or 5. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for subtilisin activity to identify amino acid residues that are critical to the activity of the molecule. See also, Hilton et al., 1996, J. Biol. Chem. 271: 4699-4708. The active site of the subtilisin or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

The relatedness between two amino acid sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows: (Identical Residues×100)/(Length of Alignment−Total Number of Gaps in Alignment).

### Non-Subtilisin Enzyme

The non-subtilisin enzyme to be combined with the subtilisin inhibitor (and the subtilisin), according to the invention, may be one or more non-subtilisin enzymes selected from the group consisting of lipase, cutinase, amylase, carbohydrase, cellulase, pectinase, pectate lyase, mannanase, arabinase, galactanase, xylanase, DNAse, perhydrolase, and oxidoreductase (oxidase, laccase, peroxidase, haloperoxidase).

The methods and compositions of the invention may include 1, 2, 3, 4, 5, 6, 7, or 8 non-subtilisin enzyme(s). A non-subtilisin enzyme is an enzyme, preferably a detergent enzyme, which is not a subtilisin.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Preferred methods and compositions of the invention comprise the subtilisin inhibitor (peptide aldehyde, hydrosulfite adducts; phenyl boronic acid or derivative thereof) and one or more non-subtilisin enzymes selected from the group consisting of: amylase; lipase/cutinase; cellulase; pectate lyase; mannanase; DNAse; perhydrolase; oxidoreductase; amylase and lipase/cutinase; amylase and cellulase; amylase and pectate lyase; amylase and mannanase; amylase and DNAse; amylase and perhydrolase; amylase and oxidoreductase; lipase/cutinase and cellulase; lipase/cutinase and pectate lyase; lipase/cutinase and mannanase; lipase/cutinase and DNAse; lipase/cutinase and perhydrolase; lipase/cutinase and oxidoreductase; cellulase and pectate lyase; cellulase and mannanase; cellulase and DNAse; cellulase and perhydrolase; cellulase and oxidoreductase; pectate lyase and mannanase; pectate lyase and DNAse; pectate lyase and perhydrolase; pectate lyase and oxidoreductase; mannanase and DNAse; mannanase and perhydrolase; mannanase and oxidoreductase; DNAse and perhydrolase; DNAse and oxidoreductase; perhydrolase and oxidoreductase; amylase and lipase/cutinase and cellulase; amylase and lipase/cutinase and pectate lyase; amylase and lipase/cutinase and mannanase; amylase and lipase/cutinase and DNAse; amylase and lipase/cutinase and perhydrolase; amylase and lipase/cutinase and oxidoreductase; amylase and cellulase and pectate lyase; amylase and cellulase and mannanase; amylase and cellulase and DNAse; amylase and cellulase and perhydrolase; amylase and cellulase and oxidoreductase; amylase and pectate lyase and mannanase; amylase and pectate lyase and DNAse; amylase and pectate lyase and perhydrolase; amylase and pectate lyase and oxidoreductase; amylase and mannanase and DNAse; amylase and mannanase and perhydrolase; amylase and mannanase and oxidoreductase; amylase and DNAse and perhydrolase; amylase and DNAse and oxidoreductase; amylase and perhydrolase and oxidoreductase; lipase/cutinase and cellulase and pectate lyase; lipase/cutinase and cellulase and mannanase; lipase/cutinase and cellulase and DNAse; lipase/cutinase and cellulase and perhydrolase; lipase/cutinase and cellulase and oxidoreductase; lipase/cutinase and pectate lyase and mannanase; lipase/cutinase and pectate lyase and DNAse; lipase/cutinase and pectate lyase and perhydrolase; lipase/cutinase and pectate lyase and oxidoreductase; lipase/cutinase and mannanase and DNAse; lipase/cutinase and mannanase and perhydrolase; lipase/cutinase and mannanase and oxidoreductase; lipase/cutinase and DNAse and perhydrolase; lipase/cutinase and DNAse and oxidoreductase; lipase/cutinase and perhydrolase and oxidoreductase; cellulase and pectate lyase and mannanase; cellulase and pectate lyase and DNAse; cellulase and pectate lyase and perhydrolase; cellulase and pectate lyase and oxidoreductase; cellulase and mannanase and DNAse; cellulase and mannanase and perhydrolase; cellulase and mannanase and oxidoreductase; cellulase and DNAse and perhydrolase; cellulase and DNAse and oxidoreductase; cellulase and perhydrolase and oxidoreductase; pectate lyase and mannanase and DNAse; pectate lyase and mannanase and perhydrolase; pectate lyase and mannanase and oxidoreductase; pectate lyase and DNAse and perhydrolase; pectate lyase and DNAse and oxidoreductase; pectate lyase and perhydrolase and oxidoreductase; mannanase and DNAse and perhydrolase; mannanase and DNAse and oxidoreductase; mannanase and perhydrolase and oxidoreductase; DNAse and perhydrolase and oxidoreductase; amylase and lipase/cutinase and cellulase and pectate lyase; amylase and lipase/cutinase and cellulase and mannanase; amylase and lipase/cutinase and cellulase and DNAse; amylase and lipase/cutinase and cellulase and perhydrolase; amylase and lipase/cutinase and cellulase and oxidoreductase; amylase and lipase/cutinase and pectate lyase and mannanase; amylase and lipase/cutinase and pectate lyase and DNAse; amylase and lipase/cutinase and pectate lyase and perhydrolase; amylase and lipase/cutinase and pectate lyase and oxidoreductase; amylase and lipase/cutinase and mannanase and DNAse; amylase and lipase/cutinase and mannanase and perhydrolase; amylase and lipase/cutinase and mannanase and oxidoreductase; amylase and lipase/cutinase and DNAse and perhydrolase; amylase and lipase/cutinase and DNAse and oxidoreductase; amylase and lipase/cutinase and perhydrolase and oxidoreductase; amylase and cellulase and pectate lyase and mannanase; amylase and cellulase and pectate lyase and DNAse; amylase and cellulase and pectate lyase and perhydrolase; amylase and cellulase and pectate lyase and oxidoreductase; amylase and cellulase and mannanase and DNAse; amylase and cellulase and mannanase and perhydrolase; amylase and cellulase and mannanase and oxidoreductase; amylase and cellulase and DNAse and perhydrolase; amylase and cellulase and DNAse and oxidoreductase; amylase and cellulase and perhydrolase and oxidoreductase; amylase and pectate lyase and mannanase and DNAse; amylase and pectate lyase and mannanase and perhydrolase; amylase and pectate lyase and mannanase and oxidoreductase; amylase and pectate lyase and DNAse and perhydrolase; amylase and pectate lyase and DNAse and oxidoreductase; amylase and pectate lyase and perhydrolase and oxidoreductase; amylase and mannanase and DNAse and perhydrolase; amylase and mannanase and DNAse and oxidoreductase; amylase and mannanase and perhydrolase and oxidoreductase; amylase and DNAse and perhydrolase and oxidoreductase; lipase/cutinase and cellulase and pectate lyase and mannanase; lipase/cutinase and cellulase and pectate lyase and DNAse; lipase/cutinase and cellulase and pectate lyase and perhydrolase; lipase/cutinase and cellulase and pectate lyase and oxidoreductase; lipase/cutinase and cellulase and mannanase and DNAse; lipase/cutinase and cellulase and mannanase and perhydrolase; lipase/cutinase and cellulase and mannanase and oxidoreductase; lipase/cutinase and cellulase and DNAse and perhydrolase; lipase/cutinase and cellulase and DNAse and oxidoreductase; lipase/cutinase and cellulase and perhydrolase and oxidoreductase; lipase/cutinase and pectate lyase and mannanase and DNAse; lipase/cutinase and pectate lyase and mannanase and perhydrolase; lipase/cutinase and pectate lyase and mannanase and oxidoreductase; lipase/cutinase and pectate lyase and DNAse and perhydrolase; lipase/cutinase and pectate lyase and DNAse and oxidoreductase; lipase/cutinase and pectate lyase and perhydrolase and oxidoreductase; lipase/cutinase and mannanase and DNAse and perhydrolase; lipase/cutinase and mannanase and DNAse and oxidoreductase; lipase/cutinase and mannanase and perhydrolase and oxidoreductase; lipase/cutinase and DNAse and perhydrolase and oxidoreductase; cellulase and pectate lyase and mannanase and DNAse; cellulase and pectate lyase and mannanase and perhydrolase; cellulase and pectate lyase and mannanase and oxidoreductase; cellulase and pectate lyase and DNAse and perhydrolase; cellulase and pectate lyase and DNAse and oxidoreductase; cellulase and pectate lyase and perhydrolase and oxidoreductase; cellulase and mannanase and DNAse and perhydrolase; cellulase and mannanase and DNAse and oxidoreductase; cellulase and mannanase and perhydrolase and oxidoreductase; cellulase and DNAse and perhydrolase and oxidoreductase; pectate lyase and mannanase and DNAse and perhydrolase; pectate lyase and mannanase and DNAse and oxidoreductase; pectate lyase and mannanase and perhydrolase and oxidoreductase; pectate lyase and DNAse and perhydrolase and oxidoreductase; mannanase and DNAse and perhydrolase and oxidoreductase; amylase and lipase/cutinase and cellulase and pectate lyase and mannanase; amylase and lipase/cutinase and cellulase and pectate lyase and DNAse; amylase and lipase/cutinase and cellulase and pectate lyase and perhydrolase; amylase and lipase/cutinase and cellulase and pectate lyase and oxidoreductase; amylase and lipase/cutinase and cellulase and mannanase and DNAse; amylase and lipase/cutinase and cellulase and mannanase and perhydrolase; amylase and lipase/cutinase and cellulase and mannanase and oxidoreductase; amylase and lipase/cutinase and cellulase and DNAse and perhydrolase; amylase and lipase/cutinase and cellulase and DNAse and oxidoreductase; amylase and lipase/cutinase and cellulase and perhydrolase and oxidoreductase; amylase and lipase/cutinase and pectate lyase and mannanase and DNAse; amylase and lipase/cutinase and pectate lyase and mannanase and perhydrolase; amylase and lipase/cutinase and pectate lyase and mannanase and oxidoreductase; amylase and lipase/cutinase and pectate lyase and DNAse and perhydrolase; amylase and lipase/cutinase and pectate lyase and DNAse and oxidoreductase; amylase and lipase/cutinase and pectate lyase and perhydrolase and oxidoreductase; amylase and lipase/cutinase and mannanase and DNAse and perhydrolase; amylase and lipase/cutinase and mannanase and DNAse and oxidoreductase; amylase and lipase/cutinase and mannanase and perhydrolase and oxidoreductase; amylase and lipase/cutinase and DNAse and perhydrolase and oxidoreductase; amylase and cellulase and pectate lyase and mannanase and DNAse; amylase and cellulase and pectate lyase and mannanase and perhydrolase; amylase and cellulase and pectate lyase and mannanase and oxidoreductase; amylase and cellulase and pectate lyase and DNAse and perhydrolase; amylase and cellulase and pectate lyase and DNAse and oxidoreductase; amylase and cellulase and pectate lyase and perhydrolase and oxidoreductase; amylase and cellulase and mannanase and DNAse and perhydrolase; amylase and cellulase and mannanase and DNAse and oxidoreductase; amylase and cellulase and mannanase and perhydrolase and oxidoreductase; amylase and cellulase and DNAse and perhydrolase and oxidoreductase; amylase and pectate lyase and mannanase and DNAse and perhydrolase; amylase and pectate lyase and mannanase and DNAse and oxidoreductase; amylase and pectate lyase and mannanase and perhydrolase and oxidoreductase; amylase and pectate lyase and DNAse and perhydrolase and oxidoreductase; amylase and mannanase and DNAse and perhydrolase and oxidoreductase; lipase/cutinase and cellulase and pectate lyase and mannanase and DNAse; lipase/cutinase and cellulase and pectate lyase and mannanase and perhydrolase; lipase/cutinase and cellulase and pectate lyase and mannanase and oxidoreductase; lipase/cutinase and cellulase and pectate lyase and

DNAse and perhydrolase; lipase/cutinase and cellulase and pectate lyase and DNAse and oxidoreductase; lipase/cutinase and cellulase and pectate lyase and perhydrolase and oxidoreductase; lipase/cutinase and cellulase and mannanase and DNAse and perhydrolase; lipase/cutinase and cellulase and mannanase and DNAse and oxidoreductase; lipase/cutinase and cellulase and mannanase and perhydrolase and oxidoreductase; lipase/cutinase and cellulase and DNAse and perhydrolase and oxidoreductase; lipase/cutinase and pectate lyase and mannanase and DNAse and perhydrolase; lipase/cutinase and pectate lyase and mannanase and DNAse and oxidoreductase; lipase/cutinase and pectate lyase and mannanase and perhydrolase and oxidoreductase; lipase/cutinase and pectate lyase and DNAse and perhydrolase and oxidoreductase; lipase/cutinase and mannanase and DNAse and perhydrolase and oxidoreductase; cellulase and pectate lyase and mannanase and DNAse and perhydrolase; cellulase and pectate lyase and mannanase and DNAse and oxidoreductase; cellulase and pectate lyase and mannanase and perhydrolase and oxidoreductase; cellulase and pectate lyase and DNAse and perhydrolase and oxidoreductase; cellulase and mannanase and DNAse and perhydrolase and oxidoreductase; pectate lyase and mannanase and DNAse and perhydrolase and oxidoreductase; amylase and lipase/cutinase and cellulase and pectate lyase and mannanase and DNAse; amylase and lipase/cutinase and cellulase and pectate lyase and mannanase and perhydrolase; amylase and lipase/cutinase and cellulase and pectate lyase and mannanase and oxidoreductase; amylase and lipase/cutinase and cellulase and pectate lyase and DNAse and perhydrolase; amylase and lipase/cutinase and cellulase and pectate lyase and DNAse and oxidoreductase; amylase and lipase/cutinase and cellulase and pectate lyase and perhydrolase and oxidoreductase; amylase and lipase/cutinase and cellulase and mannanase and DNAse and perhydrolase; amylase and lipase/cutinase and cellulase and mannanase and DNAse and oxidoreductase; amylase and lipase/cutinase and cellulase and mannanase and perhydrolase and oxidoreductase; amylase and lipase/cutinase and cellulase and DNAse and perhydrolase and oxidoreductase; amylase and lipase/cutinase and pectate lyase and mannanase and DNAse and perhydrolase; amylase and lipase/cutinase and pectate lyase and mannanase and DNAse and oxidoreductase; amylase and lipase/cutinase and pectate lyase and mannanase and perhydrolase and oxidoreductase; amylase and lipase/cutinase and pectate lyase and DNAse and perhydrolase and oxidoreductase; amylase and lipase/cutinase and mannanase and DNAse and perhydrolase and oxidoreductase; amylase and cellulase and pectate lyase and mannanase and DNAse and perhydrolase; amylase and cellulase and pectate lyase and mannanase and DNAse and oxidoreductase; amylase and cellulase and pectate lyase and mannanase and perhydrolase and oxidoreductase; amylase and cellulase and pectate lyase and DNAse and perhydrolase and oxidoreductase; amylase and cellulase and mannanase and DNAse and perhydrolase and oxidoreductase; amylase and pectate lyase and mannanase and DNAse and perhydrolase and oxidoreductase; lipase/cutinase and cellulase and pectate lyase and mannanase and DNAse and perhydrolase; lipase/cutinase and cellulase and pectate lyase and mannanase and DNAse and oxidoreductase; lipase/cutinase and cellulase and pectate lyase and mannanase and perhydrolase and oxidoreductase; lipase/cutinase and cellulase and pectate lyase and DNAse and perhydrolase and oxidoreductase; lipase/cutinase and cellulase and mannanase and DNAse and perhydrolase and oxidoreductase; lipase/cutinase and pectate lyase and mannanase and DNAse and perhydrolase and oxidoreductase; cellulase and pectate lyase and mannanase and DNAse and perhydrolase and oxidoreductase; amylase and lipase/cutinase and cellulase and pectate lyase and mannanase and DNAse and perhydrolase; amylase and lipase/cutinase and cellulase and pectate lyase and mannanase and DNAse and oxidoreductase; amylase and lipase/cutinase and cellulase and pectate lyase and mannanase and perhydrolase and oxidoreductase; amylase and lipase/cutinase and cellulase and pectate lyase and DNAse and perhydrolase and oxidoreductase; amylase and lipase/cutinase and cellulase and mannanase and DNAse and perhydrolase and oxidoreductase; amylase and lipase/cutinase and pectate lyase and mannanase and DNAse and perhydrolase and oxidoreductase; amylase and cellulase and pectate lyase and mannanase and DNAse and perhydrolase and oxidoreductase; lipase/cutinase and cellulase and pectate lyase and mannanase and DNAse and perhydrolase and oxidoreductase; and amylase and lipase/cutinase and cellulase and pectate lyase and mannanase and DNAse and perhydrolase and oxidoreductase.

### Lipase/Cutinase

Suitable lipases and cutinases include those of bacterial or fungal origin. Chemically modified or protein engineered mutants are included. Examples include lipase from Thermomyces, e.g., from T. lanuginosus (previously named Humicola lanuginosa) as described in EP 258068 and EP 305216, cutinase from Humicola, e.g. H. insolens as described in WO 96/13580, a Pseudomonas lipase, e.g., from P. alcaligenes or P. pseudoalcaligenes (EP 218272), P. cepacia (EP 331376), P. stutzeri (GB 1372034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., Biochemica et Biophysica Acta, (1993), 1131, 253-360), B. stearothermophilus (JP 64/744992) or B. pumilus (WO 91/16422), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in WO 92/05249, WO 94/01541, EP 407225, EP 260105, WO 95/35381, WO 96/00292, WO 95/30744, WO 94/25578, WO 95/14783, WO 95/22615, WO 97/04079, WO 97/07202, WO 00/060063, WO 07/087508 and WO 09/109500.

Preferred commercially available lipase enzymes include Lipolase™, Lipolase Ultra™, and Lipex™; Lecitase™, Lipolex™; Lipoclean™, Lipoprime™ (Novozymes NS). Other commercially available lipases include Lumafast (Genencor Int Inc); Lipomax (Gist-Brocades/Genencor Int Inc) and Bacillus sp lipase from Solvay.

### Carbohydrase

A carbohydrase is a general term for enzymes that cleave carbohydrates. In general carbohydrases are named after the substrates they act on, for example amylases act on amylase and cellulases act on cellulose. Many carbohydrases have found use in cleaning and laundry applications, such as amylase, cellulase, pectinase, pectate lyase, mannanase, arabinase, galactanase and xylanase, and all these can be applied in the liquid composition.

### Amylase

Suitable amylases include those of bacterial or fungal origin. Chemically modified or protein engineered mutants are included. Amylases include, for example, α-amylases obtained from Bacillus, e.g., a special strain of Bacillus licheniformis, described in more detail in GB 1,296,839.

Examples of suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193. Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

### M197T;

### H156Y+A181T+N190F+A209V+Q264S; or

### G48A+T491+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

### N128C+K178L+T182G+Y305R+G475K;

### N128C+K178L+T182G+F202Y+Y305R+D319T+G475K;

### S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particularly preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Stainzyme™, Stainzyme Plus™, Amplify™, Resilience™, Everest™, Duramyl™, Termamyl™, Termamyl Ultra™, Natalase™, Fungamyl™ and BAN™ (Novozymes NS), Rapidase™ and Purastar™/Effectenz™, Powerase™ and Preferenz S100 (from Genencor International Inc./DuPont).

### Lyases

The lyase may be a pectate lyase derived from Bacillus, particularly B. licherniformis or B. agaradhaerens, or a variant derived of any of these, e.g. as described in U.S. Pat. No. 6,124,127, WO 99/027083, WO 99/027084, WO 02/006442, WO 02/092741, WO 03/095638, Commercially available pectate lyases are XPect™, Pectawash™, and Pectaway™ (Novozymes NS).

### Mannanase

The mannanase may be an alkaline mannanase of Family 5 or 26. It belongs It may be a wild-type from Bacillus or Humicola, particularly B. agaradhaerens, B. licheniformis, B. halodurans, B. clausii, or H. insolens. Suitable mannanases are described in WO 99/064619. A commercially available mannanase is Mannaway™ (Novozymes NS).

### Cellulase

Suitable cellulases may be of bacterial or fungal origin. Chemically or genetically modified mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, US

Commercially available cellulases include Carezyme™, Celluzyme™, Celluclean™, Celluclast™, Endolase™, Renozyme™, Whitezyme™ (Novozymes NS); Clazinase™, Puradax, Puradax HA, and Puradax EG (available from Genencor) and KAC-500(B)™ (Kao Corporation).

### Peroxidases/Oxidases

Suitable peroxidases are comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

The peroxidases also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

Suitable oxidases include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus. A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Deoxyribonuclease (DNase)

Suitable deoxyribonucleases (DNases) are any enzyme that catalyzes the hydrolytic cleavage of phosphodiester linkages in the DNA backbone, thus degrading DNA. According to the invention, a DNase which is obtainable from a bacterium is preferred; in particular a DNase which is obtainable from a Bacillus is preferred; in particular a DNase which is obtainable from Bacillus subtilis or Bacillus licheniformis is preferred. Examples of such DNases are described in patent application WO 2011/098579 or in PCT/EP2013/075922.

### Perhydrolase

Suitable perhydrolases are capable of catalyzing a perhydrolysis reaction that results in the production of a peracid from a carboxylic acid ester (acyl) substrate in the presence of a source of peroxygen (e.g., hydrogen peroxide). While many enzymes perform this reaction at low levels, perhydrolases exhibit a high perhydrolysis:hydrolysis ratio, often greater than 1. Suitable perhydrolases may be of plant, bacterial or fungal origin. Chemically modified or protein engineered mutants are included.

Examples of useful perhydrolases include naturally occurring Mycobacterium perhydrolase enzymes, or variants thereof. An exemplary enzyme is derived from Mycobacterium smegmatis. Such enzyme, its enzymatic properties, its structure, and variants thereof, are described in WO 2005/056782, WO 2008/063400, US 2008/145353, and US2007167344.

### Subtilisin Inhibitor

The subtilisin inhibitor used in the methods and compositions of the invention is a phenyl boronic acid or a derivative thereof; or a peptide aldehyde, a hydrosulfite adduct thereof, or a peptide methyl ketone. The methyl group is optionally halogen-substituted, and the peptide optionally has an N-terminal protection group.

Preferably, the subtilisin inhibitor is a peptide aldehyde, a hydrosulfite adduct thereof, or a peptide methyl ketone.

### Peptide Aldehyde or Ketone

The inhibitor may have the formula: P-(A)_{y}-L-(B)ₓ—B⁰—R* wherein:

R* is H (hydrogen), CH₃, CX₃, CHX₂, or CH₂X. Preferably, R*═H so that the inhibitor is a peptide aldehyde with the formula P-(A)_{y}-L-(B)ₓ—B⁰—H;

X is a halogen atom, particularly F (fluorine);

B⁰ is a single amino acid residue with L- or D-configuration of the formula —NH—CH(R)—C(═O)—;

x is 1, 2 or 3;

Bₓ is independently a single amino acid residue, each connected to the next B or to B⁰ via its C-terminal;

L is absent or independently a linker group of the formula —C(═O)—, —C(═O)—C(═O)—, —C(═S)—, —C(═S)—C(═S)— or —C(═S)—C(═O)—;

A is absent if L is absent or is independently a single amino acid residue connected to L via the N-terminal of the amino acid;

P is selected from the group consisting of hydrogen or if L is absent an N-terminal protection group;

y is 0, 1, or 2,

R is independently selected from the group consisting of C₁₋₆ alkyl, C₆₋₁₀ aryl or C₇₋₁₀ arylalkyl optionally substituted with one or more, identical or different, substituent's R′;

R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂; and

R″ is a C₁₋₆ alkyl group.

x may be 1, 2 or 3 and therefore B may be 1, 2 or 3 amino acid residues respectively. Thus, B may represent B¹, B²—B¹ or B³—B²—B¹, where B³, B² and B¹ each represent one amino acid residue. y may be 0, 1 or 2 and therefore A may be absent, or 1 or 2 amino acid residues respectively having the formula A¹ or A²-A¹ wherein A² and A¹ each represent one amino acid residue.

B⁰ may be a single amino acid residue with L- or D-configuration, which is connected to H via the C-terminal of the amino acid. B⁰ has the formula —NH—CH(R)—C(═O)—, wherein R is a C₁₋₆ alkyl, C₆₋₁₀ aryl or C₇₋₁₀ arylalkyl side chain, such as methyl, ethyl, propyl, isopropyl, butyl, isobutyl, phenyl or benzyl, and wherein R may be optionally substituted with one or more, identical or different, substituent's R′. Particular examples of B⁰ are the D- or L-form of arginine (Arg), 3,4-dihydroxyphenylalanine, isoleucine (Ile), leucine (Leu), methionine (Met), norleucine (Nle), norvaline (Nva), phenylalanine (Phe), m-tyrosine, p-tyrosine (Tyr) and valine (Val). A particular embodiment is when B⁰ is leucine, methionine, phenylalanine, p-tyrosine and valine.

B¹, which is connected to B⁰ via the C-terminal of the amino acid, may be an aliphatic, hydrophobic and/or neutral amino acid. Examples of B¹ are alanine (Ala), cysteine (Cys), glycine (Gly), isoleucine (Ile), leucine (Leu), norleucine (Nle), norvaline (Nva), proline (Pro), serine (Ser), threonine (Thr) and valine (Val). Particular examples of B¹ are alanine, glycine, isoleucine, leucine and valine. A particular embodiment is when B¹ is alanine, glycine or valine.

If present, B², which is connected to B¹ via the C-terminal of the amino acid, may be an aliphatic, hydrophobic, neutral and/or polar amino acid. Examples of B² are alanine (Ala), arginine (Arg), capreomycidine (Cpd), cysteine (Cys), glycine (Gly), isoleucine (Ile), leucine (Leu), norleucine (Nie), norvaline (Nva), phenylalanine (Phe), proline (Pro), serine (Ser), threonine (Thr), and valine (Val). Particular examples of B² are alanine, arginine, capreomycidine, glycine, isoleucine, leucine, phenylalanine and valine. A particular embodiment is when B² is arginine, glycine, leucine, phenylalanine or valine.

B³, which if present is connected to B² via the C-terminal of the amino acid, may be a large, aliphatic, aromatic, hydrophobic and/or neutral amino acid. Examples of B³ are isoleucine

(Ile), leucine (Leu), norleucine (Nie), norvaline (Nva), phenylalanine (Phe), phenylglycine, tyrosine (Tyr), tryptophan (Trp) and valine (Val). Particular examples of B³ are leucine, phenylalanine, tyrosine and tryptophan.

The linker group L may be absent or selected from the group consisting of —C(═O)—, —C(═O)—O(═O)—, —O(═S)—, —O(═S)—O(═S)— or —O(═S)—O(═O)—. Particular embodiments of the invention are when L is absent or L is a carbonyl group —O(═O)—.

A¹, which if present is connected to L via the N-terminal of the amino acid, may be an aliphatic, aromatic, hydrophobic, neutral and/or polar amino acid. Examples of A¹ are alanine (Ala), arginine (Arg), capreomycidine (Cpd), glycine (Gly), isoleucine (Ile), leucine (Leu), norleucine (Nle), norvaline (Nva), phenylalanine (Phe), threonine (Thr), tyrosine (Tyr), tryptophan (Trp) and valine (Val). Particular examples of A¹ are alanine, arginine, glycine, leucine, phenylalanine, tyrosine, tryptophan and valine. A particular embodiment is when B² is leucine, phenylalanine, tyrosine or tryptophan.

The A² residue, which if present is connected to A¹ via the N-terminal of the amino acid, may be a large, aliphatic, aromatic, hydrophobic and/or neutral amino acid. Examples of A² are arginine (Arg), isoleucine (Ile), leucine (Leu), norleucine (Nle), norvaline (Nva), phenylalanine (Phe), phenylglycine, Tyrosine (Tyr), tryptophan (Trp) and valine (Val). Particular examples of A² are phenylalanine and tyrosine.

The N-terminal protection group P (if present) may be selected from formyl, acetyl (Ac), benzoyl (Bz), trifluoroacetyl, methoxysuccinyl, aromatic and aliphatic urethane protecting groups such as fluorenylmethyloxycarbonyl (Fmoc), methoxycarbonyl (Moc), (fluoromethoxy)carbonyl, benzyloxycarbonyl (Cbz), t-butyloxycarbonyl (Boc) and adamantyloxycarbonyl; p-methoxybenzyl carbonyl, benzyl (Bn), p-methoxybenzyl (PMB), p-methoxyphenyl (PMP), methoxyacetyl, methylamino carbonyl, methylsulfonyl, ethylsulfonyl, benzylsulfonyl, methylphosphoramidyl (MeOP(OH)(═O)) and benzylphosphoramidyl (PhCH₂OP(OH)(═O)).

In the case of a tripeptide aldehyde with a protection group (i.e. x=2, L is absent and A is absent), P is preferably acetyl, methoxycarbonyl, benzyloxycarbonyl, methylamino carbonyl, methylsulfonyl, benzylsulfonyl and benzylphosphoramidyl. In the case of a tetrapeptide aldehyde with a protection group (i.e. x=3, L is absent and A is absent), P is preferably acetyl, methoxycarbonyl, methylsulfonyl, ethylsulfonyl and methylphosphoramidyl.

Suitable peptide aldehydes are described in WO94/04651, WO95/25791, WO98/13458, WO98/13459, WO98/13460, WO98/13461, WO98/13462, WO07/141736, WO07/145963, WO09/118375, WO10/055052 and WO11/036153. More particularly, the peptide aldehyde may be Cbz-Arg-Ala-Tyr-H, Ac-Gly-Ala-Tyr-H, Cbz-Gly-Ala-Tyr-H, Cbz-Gly-Ala-Tyr-CF₃, Cbz-Gly-Ala-Leu-H, Cbz-Val-Ala-Leu-H, Cbz-Val-Ala-Leu-CF₃, Moc-Val-Ala-Leu-CF₃, Cbz-Gly-Ala-Phe-H, Cbz-Gly-Ala-Phe-CF₃, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO₂-Phe-Gly-Ala-Leu-H, MeSO₂-Val-Ala-Leu-H, PhCH₂O—P(OH)(O)-Val-Ala-Leu-H, EtSO₂-Phe-Gly-Ala-Leu-H, PhCH₂SO₂-Val-Ala-Leu-H, PhCH₂O—P(OH)(O)-Leu-Ala-Leu-H, PhCH₂O—P(OH)(O)-Phe-Ala-Leu-H, or MeO—P(OH)(O)-Leu-Gly-Ala-Leu-H. A preferred inhibitor for use in the liquid composition of the invention is Cbz-Gly-Ala-Tyr-H, or a hydrosulfite adduct thereof, wherein Cbz is benzyloxycarbonyl.

Further examples of such peptide aldehydes include α-MAPI, β-MAPI, Phe-C(═O)-Arg-Val-Tyr-H, Phe-C(═O)-Gly-Gly-Tyr-H, Phe-C(═O)-Gly-Ala-Phe-H, Phe-C(═O)-Gly-Ala-Tyr-H, Phe-C(═O)-Gly-Ala-L-H, Phe-C(═O)-Gly-Ala-Nva-H, Phe-C(═O)-Gly-Ala-Nle-H, Tyr-C(═O)-Arg-Val-Tyr-H, Tyr-C(═O)-Gly-Ala-Tyr-H, Phe-C(═S)-Arg-Val-Phe-H, Phe-C(═S)-Arg-Val-Tyr-H, Phe-C(═S)-Gly-Ala-Tyr-H, Antipain, GE20372A, GE20372B, Chymostatin A, Chymostatin B, and Chymostatin C.

### Hydrosulfite Adduct

The subtilisin inhibitor may be a hydrosulfite adduct of the peptide aldehyde described above, e.g. as described in WO 2013/004636. The adduct may have the formula P-(A)_{y}-L-(B)ₓ—N(H)—CHR—CH(OH)—SO₃M, wherein P, A, y, L, B, x and R are defined as above, and M is H or an alkali metal, preferably Na or K. A preferred embodiment is a hydrosulfite adduct wherein P=Cbz, B²=Gly; B¹=Ala; B⁰=Tyr (so R=PhCH₂, R′=OH), x=2, y=0, L=A=absent and M=Na.

### Peptide Aldehyde or Hydrosulfite Adduct

The inhibitor may be an aldehyde having the formula P—B²—B¹—B⁰—H or an adduct having the formula P—B²—B¹—N(H)—CHR—CHOH—SO₃M, wherein

a) H is hydrogen;
b) B⁰ is a single amino acid residue with L- or D-configuration of the formula —NH—CH(R)—C(═O)—;
c) B¹ and B² are independently single amino acid residues;
d) R is independently selected from the group consisting of C₁₋₆ alkyl, C₆₋₁₀ aryl or C₇₋₁₀ arylalkyl optionally substituted with one or more, identical or different, substituent's R′;
e) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂;
f) R″ is a C₁₋₆ alkyl group; and
g) P is an N-terminal protection group.
Constituents b) to g) may be selected as described above.

### Phenyl Boronic Acids

The subtilisin inhibitor may be phenyl boronic acid or a derivative thereof.

In an embodiment of the invention, the phenyl boronic acid derivative is of the following formula:

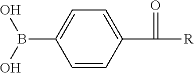

wherein R is selected from the group consisting of hydrogen, hydroxy, C₁-C₆ alkyl, substituted C₁-C₆ alkyl, C₁-C₆ alkenyl and substituted C₁-C₆ alkenyl. Preferably, R is hydrogen, CH₃, CH₃CH₂ or CH₃CH₂CH₂.

In a preferred embodiment, the subtilisin inhibitor (phenyl boronic acid derivative) is 4-formyl-phenyl-boronic acid (4-FPBA).

In another particular embodiment, the subtilisin inhibitor is selected from the group consisting of:

thiophene-2 boronic acid, thiophene-3 boronic acid, acetamidophenyl boronic acid, benzofuran-2 boronic acid, naphtalene-1 boronic acid, naphtalene-2 boronic acid, 2-FPBA, 3-FBPA, 4-FPBA, 1-thianthrene boronic acid, 4-dibenzofuran boronic acid, 5-methylthiophene-2 boronic, acid, thionaphtrene boronic acid, furan-2 boronic acid, furan-3 boronic acid, 4,4 biphenyl-diborinic acid, 6-hydroxy-2-naphtalene, 4-(methylthio) phenyl boronic acid, 4 (trimethyl-silyl)phenyl boronic acid, 3-bromothiophene boronic acid, 4-methylthiophene boronic acid, 2-naphtyl boronic acid, 5-bromothiphene boronic acid, 5-chlorothiophene boronic acid, dimethylthiophene boronic acid, 2-bromophenyl boronic acid, 3-chlorophenyl boronic acid, 3-methoxy-2-thiophene, p-methyl-phenylethyl boronic acid, 2-thianthrene boronic acid, di-benzothiophene boronic acid, 4-carboxyphenyl boronic acid, 9-anthryl boronic acid, 3,5 dichlorophenyl boronic, acid, diphenyl boronic acidanhydride, o-chlorophenyl boronic acid, p-chlorophenyl boronic acid, m-bromophenyl boronic acid, p-bromophenyl boronic acid, p-flourophenyl boronic acid, p-tolyl boronic acid, o-tolyl boronic acid, octyl boronic acid, 1,3,5 trimethylphenyl boronic acid, 3-chloro-4-flourophenyl boronic acid, 3-aminophenyl boronic acid, 3,5-bis-(triflouromethyl) phenyl boronic acid, 2,4 dichlorophenyl boronic acid, 4-methoxyphenyl boronic acid.

Further boronic acid derivatives suitable as subtilisin inhibitors in the detergent composition are described in U.S. Pat. No. 4,963,655, U.S. Pat. No. 5,159,060, WO 95/12655, WO 95/29223, WO 92/19707, WO 94/04653, WO 94/04654, U.S. Pat. No. 5,442,100, U.S. Pat. No. 5,488,157 and U.S. Pat. No. 5,472,628.

### Liquid Detergent Composition

The liquid detergent composition has a physical form, which is not solid (or gas). It may be a pourable liquid, a pourable gel or a non-pourable gel. It may be either isotropic or structured, preferably isotropic. It may be a formulation useful for washing in automatic washing machines or for hand washing.

The liquid detergent composition may be aqueous, typically containing at least 20% by weight and up to 95% water, such as up to 70% water, up to 50% water, up to 40% water, up to 30% water, or up to 20% water. Other types of liquids, including without limitation, alkanols, amines, diols, ethers and polyols may be included in an aqueous liquid detergent. An aqueous liquid detergent may contain from 0-30% organic solvent. A liquid detergent may even be non-aqueous, wherein the water content is below 10%, preferably below 5%.

Detergent ingredients can be separated physically from each other by compartments in water dissolvable pouches. Thereby negative storage interaction between components can be avoided. Different dissolution profiles of each of the compartments can also give rise to delayed dissolution of selected components in the wash solution.

The detergent composition may take the form of a unit dose product. A unit dose product is the packaging of a single dose in a non-reusable container. It is increasingly used in detergents for laundry and dish wash. A detergent unit dose product is the packaging (e.g., in a pouch made from a water soluble film) of the amount of detergent used for a single wash.

Pouches can be of any form, shape and material which is suitable for holding the composition, e.g., without allowing the release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be a blend compositions comprising hydrolytically degradable and water soluble polymer blends such as polyactide and polyvinyl alcohol (known under the Trade reference M8630 as sold by Chris Craft In. Prod. Of Gary, Ind., US) plus plasticizers like glycerol, ethylene glycerol, Propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids (see e.g., US 2009/0011970).

The choice of detergent components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

The choice of additional components is within the skill of the artisan and includes conventional ingredients, including the exemplary non-limiting components set forth below.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually contain from about 0.1% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent will usually contain from about 0.1% to about 20% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N, N-bis(2-hydroxyethyl)amine oxide, fatty acid alkanolamides and ethoxylated fatty acid alkanolamides, and combinations thereof.

When included therein the detergent will usually contain from about 0.1% to about 10% by weight of a zwitterionic surfactant. Non-limiting examples of zwitterionic surfactants include betaine, alkyldimethylbetaine, sulfobetaine, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see for example review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg ions. Any builder and/or co-builder known in the art for use in laundry detergents may be utilized. Non-limiting examples of builders include citrates, zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-50% by weight, such as about 0.5% to about 10%, of a detergent co-builder, or a mixture thereof. The detergent composition may include a co-builder alone, or in combination with a builder, for example a citrate builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N′, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575 and U.S. Pat. No. 5,955,415. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/03274, WO 2005/03275, WO 2005/03276 and EP 1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g., WO 2007/087257 and WO 2007/087243.

### Additional Enzymes

The liquid detergent composition may comprise additional enzymes using other formulation technologies, such as microcapsules (e.g., as described in PCT/EP2014/059017 or WO 1997/024177), particles, or enzymatic water soluble films (e.g., as described in PCT/US2014/027603, PCT/US2014/027462, or WO 2013/148492).

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants—

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents—

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Agent—

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Soil Release Polymers—

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalate based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Agents—

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Rheology Modifiers are structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of the composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Bleaching Systems

Due to the incompatibility of the components there are still only few examples of liquid detergents combining bleach and enzymes (e.g., U.S. Pat. No. 5,275,753 or WO 99/00478). The detergent may contain 0-50% of a bleaching system. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone®, and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO 98/17767. A particular family of bleach activators of interest was disclosed in EP624154 and particularly preferred in that family is acetyl triethyl citrate (ATC). ATC or a short chain triglyceride like triacetin has the advantage that it is environmental friendly as it eventually degrades into citric acid and alcohol. Furthermore acetyl triethyl citrate and triacetin has a good hydrolytical stability in the product upon storage and it is an efficient bleach activator. Finally ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

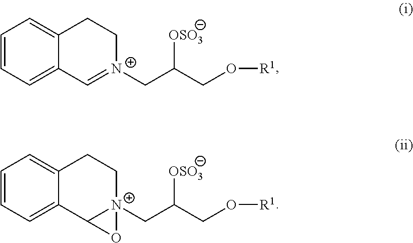

and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g., in WO 2007/087258, WO 2007/087244, WO 2007/087259 and WO 2007/087242. Suitable photobleaches may for example be sulfonated zinc phthalocyanine.

### Formulation of Detergent Products

The liquid detergent composition of the invention may be in any convenient form, e.g., a pouch having one or more compartments, a gel, or a regular, compact or concentrated liquid detergent (see e.g., WO 2009/098660 or WO 2010/141301).

Pouches can be configured as single or multi compartments. It can be of any form, shape and material which is suitable for holding the composition, e.g., without allowing release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids.

Detergent ingredients can be separated physically from each other by compartments in water dissolvable pouches. Thereby negative storage interaction between components can be avoided. Different dissolution profiles of each of the compartments can also give rise to delayed dissolution of selected components in the wash solution.

### Compositions, Methods and Uses

As described in the above paragraphs, the present invention provides a method for improving the storage stability of a non-subtilisin enzyme in a liquid detergent composition containing a subtilisin and a non-subtilisin, comprising preparing a liquid detergent composition containing a subtilisin, a non-subtilisin, and more than a concentration C of a subtilisin inhibitor; wherein C is determined as the concentration of inhibitor where the relative residual activity of the subtilisin is increased by 25% of the increase from no inhibitor to C, when the concentration of the subtilisin inhibitor is increased from C to two times C, after incubation for one week at 40° C. in Detergent M. Preferably, The relative storage stability improvement of the non-subtilisin from C to two times C is higher than the relative storage stability improvement of the subtilisin.

In an embodiment, C is 10 mg/L, or C corresponds to a stoichiometric ratio of inhibitor:subtilisin of 3:1. The liquid detergent composition may contain up to 100 mg/L of the subtilisin inhibitor.

In an embodiment, the liquid detergent composition is prepared by combining a subtilisin composition and a non-subtilisin enzyme composition, wherein the non-subtilisin enzyme composition comprises part of, or all of, the subtilisin inhibitor. Preferably, the non-subtilisin enzyme composition comprises at least 50% of the subtilisin inhibitor.

In an embodiment, the non-subtilisin enzyme is one or more enzymes selected from the group consisting of amylase, lipase, cellulase, pectinase, mannanase, DNAse, perhydrolase, and oxidoreductase. Preferably the one or more non-subtilisin enzyme is selected from the group consisting of: amylase; lipase/cutinase; cellulase; pectate lyase; mannanase; DNAse; perhydrolase; oxidoreductase; amylase and lipase/cutinase; amylase and cellulase; amylase and pectate lyase; amylase and mannanase; amylase and DNAse; amylase and perhydrolase; amylase and oxidoreductase; lipase/cutinase and cellulase; lipase/cutinase and pectate lyase; lipase/cutinase and mannanase; lipase/cutinase and DNAse; lipase/cutinase and perhydrolase; lipase/cutinase and oxidoreductase; cellulase and pectate lyase; cellulase and mannanase; cellulase and DNAse; cellulase and perhydrolase; cellulase and oxidoreductase; pectate lyase and mannanase; pectate lyase and DNAse; pectate lyase and perhydrolase; pectate lyase and oxidoreductase; mannanase and DNAse; mannanase and perhydrolase; mannanase and oxidoreductase; DNAse and perhydrolase; DNAse and oxidoreductase; perhydrolase and oxidoreductase; amylase and lipase/cutinase and cellulase; amylase and lipase/cutinase and pectate lyase; amylase and lipase/cutinase and mannanase; amylase and lipase/cutinase and DNAse; amylase and lipase/cutinase and perhydrolase; amylase and lipase/cutinase and oxidoreductase; amylase and cellulase and pectate lyase; amylase and cellulase and mannanase; amylase and cellulase and DNAse; amylase and cellulase and perhydrolase; amylase and cellulase and oxidoreductase; amylase and pectate lyase and mannanase; amylase and pectate lyase and DNAse; amylase and pectate lyase and perhydrolase; amylase and pectate lyase and oxidoreductase; amylase and mannanase and DNAse; amylase and mannanase and perhydrolase; amylase and mannanase and oxidoreductase; amylase and DNAse and perhydrolase; amylase and DNAse and oxidoreductase; amylase and perhydrolase and oxidoreductase; lipase/cutinase and cellulase and pectate lyase; lipase/cutinase and cellulase and mannanase; lipase/cutinase and cellulase and DNAse; lipase/cutinase and cellulase and perhydrolase; lipase/cutinase and cellulase and oxidoreductase; lipase/cutinase and pectate lyase and mannanase; lipase/cutinase and pectate lyase and DNAse; lipase/cutinase and pectate lyase and perhydrolase; lipase/cutinase and pectate lyase and oxidoreductase; lipase/cutinase and mannanase and DNAse; lipase/cutinase and mannanase and perhydrolase; lipase/cutinase and mannanase and oxidoreductase; lipase/cutinase and DNAse and perhydrolase; lipase/cutinase and DNAse and oxidoreductase; lipase/cutinase and perhydrolase and oxidoreductase; cellulase and pectate lyase and mannanase; cellulase and pectate lyase and DNAse; cellulase and pectate lyase and perhydrolase; cellulase and pectate lyase and oxidoreductase; cellulase and mannanase and DNAse; cellulase and mannanase and perhydrolase; cellulase and mannanase and oxidoreductase; cellulase and DNAse and perhydrolase; cellulase and DNAse and oxidoreductase; cellulase and perhydrolase and oxidoreductase; pectate lyase and mannanase and DNAse; pectate lyase and mannanase and perhydrolase; pectate lyase and mannanase and oxidoreductase; pectate lyase and DNAse and perhydrolase; pectate lyase and DNAse and oxidoreductase; pectate lyase and perhydrolase and oxidoreductase; mannanase and DNAse and perhydrolase; mannanase and DNAse and oxidoreductase; mannanase and perhydrolase and oxidoreductase; DNAse and perhydrolase and oxidoreductase; amylase and lipase/cutinase and cellulase and pectate lyase; amylase and lipase/cutinase and cellulase and mannanase; amylase and lipase/cutinase and cellulase and DNAse; amylase and lipase/cutinase and cellulase and perhydrolase; amylase and lipase/cutinase and cellulase and oxidoreductase; amylase and lipase/cutinase and pectate lyase and mannanase; amylase and lipase/cutinase and pectate lyase and DNAse; amylase and lipase/cutinase and pectate lyase and perhydrolase; amylase and lipase/cutinase and pectate lyase and oxidoreductase; amylase and lipase/cutinase and mannanase and DNAse; amylase and lipase/cutinase and mannanase and perhydrolase; amylase and lipase/cutinase and mannanase and oxidoreductase; amylase and lipase/cutinase and DNAse and perhydrolase; amylase and lipase/cutinase and DNAse and oxidoreductase; amylase and lipase/cutinase and perhydrolase and oxidoreductase; amylase and cellulase and pectate lyase and mannanase; amylase and cellulase and pectate lyase and DNAse; amylase and cellulase and pectate lyase and perhydrolase; amylase and cellulase and pectate lyase and oxidoreductase; amylase and cellulase and mannanase and DNAse; amylase and cellulase and mannanase and perhydrolase; amylase and cellulase and mannanase and oxidoreductase; amylase and cellulase and DNAse and perhydrolase; amylase and cellulase and DNAse and oxidoreductase; amylase and cellulase and perhydrolase and oxidoreductase; amylase and pectate lyase and mannanase and DNAse; amylase and pectate lyase and mannanase and perhydrolase; amylase and pectate lyase and mannanase and oxidoreductase; amylase and pectate lyase and DNAse and perhydrolase; amylase and pectate lyase and DNAse and oxidoreductase; amylase and pectate lyase and perhydrolase and oxidoreductase; amylase and mannanase and DNAse and perhydrolase; amylase and mannanase and DNAse and oxidoreductase; amylase and mannanase and perhydrolase and oxidoreductase; amylase and DNAse and perhydrolase and oxidoreductase; lipase/cutinase and cellulase and pectate lyase and mannanase; lipase/cutinase and cellulase and pectate lyase and DNAse; lipase/cutinase and cellulase and pectate lyase and perhydrolase; lipase/cutinase and cellulase and pectate lyase and oxidoreductase; lipase/cutinase and cellulase and mannanase and DNAse; lipase/cutinase and cellulase and mannanase and perhydrolase; lipase/cutinase and cellulase and mannanase and oxidoreductase; lipase/cutinase and cellulase and DNAse and perhydrolase; lipase/cutinase and cellulase and DNAse and oxidoreductase; lipase/cutinase and cellulase and perhydrolase and oxidoreductase; lipase/cutinase and pectate lyase and mannanase and DNAse; lipase/cutinase and pectate lyase and mannanase and perhydrolase; lipase/cutinase and pectate lyase and mannanase and oxidoreductase; lipase/cutinase and pectate lyase and DNAse and perhydrolase; lipase/cutinase and pectate lyase and DNAse and oxidoreductase; lipase/cutinase and pectate lyase and perhydrolase and oxidoreductase; lipase/cutinase and mannanase and DNAse and perhydrolase; lipase/cutinase and mannanase and DNAse and oxidoreductase; lipase/cutinase and mannanase and perhydrolase and oxidoreductase; lipase/cutinase and DNAse and perhydrolase and oxidoreductase; cellulase and pectate lyase and mannanase and DNAse; cellulase and pectate lyase and mannanase and perhydrolase; cellulase and pectate lyase and mannanase and oxidoreductase; cellulase and pectate lyase and DNAse and perhydrolase; cellulase and pectate lyase and DNAse and oxidoreductase; cellulase and pectate lyase and perhydrolase and oxidoreductase; cellulase and mannanase and DNAse and perhydrolase; cellulase and mannanase and DNAse and oxidoreductase; cellulase and mannanase and perhydrolase and oxidoreductase; cellulase and DNAse and perhydrolase and oxidoreductase; pectate lyase and mannanase and DNAse and perhydrolase; pectate lyase and mannanase and DNAse and oxidoreductase; pectate lyase and mannanase and perhydrolase and oxidoreductase; pectate lyase and DNAse and perhydrolase and oxidoreductase; mannanase and DNAse and perhydrolase and oxidoreductase; amylase and lipase/cutinase and cellulase and pectate lyase and mannanase; amylase and lipase/cutinase and cellulase and pectate lyase and DNAse; amylase and lipase/cutinase and cellulase and pectate lyase and perhydrolase; amylase and lipase/cutinase and cellulase and pectate lyase and oxidoreductase; amylase and lipase/cutinase and cellulase and mannanase and DNAse; amylase and lipase/cutinase and cellulase and mannanase and perhydrolase; amylase and lipase/cutinase and cellulase and mannanase and oxidoreductase; amylase and lipase/cutinase and cellulase and DNAse and perhydrolase; amylase and lipase/cutinase and cellulase and DNAse and oxidoreductase; amylase and lipase/cutinase and cellulase and perhydrolase and oxidoreductase; amylase and lipase/cutinase and pectate lyase and mannanase and DNAse; amylase and lipase/cutinase and pectate lyase and mannanase and perhydrolase; amylase and lipase/cutinase and pectate lyase and mannanase and oxidoreductase; amylase and lipase/cutinase and pectate lyase and DNAse and perhydrolase; amylase and lipase/cutinase and pectate lyase and DNAse and oxidoreductase; amylase and lipase/cutinase and pectate lyase and perhydrolase and oxidoreductase; amylase and lipase/cutinase and mannanase and DNAse and perhydrolase; amylase and lipase/cutinase and mannanase and DNAse and oxidoreductase; amylase and lipase/cutinase and mannanase and perhydrolase and oxidoreductase; amylase and lipase/cutinase and DNAse and perhydrolase and oxidoreductase; amylase and cellulase and pectate lyase and mannanase and DNAse; amylase and cellulase and pectate lyase and mannanase and perhydrolase; amylase and cellulase and pectate lyase and mannanase and oxidoreductase; amylase and cellulase and pectate lyase and DNAse and perhydrolase; amylase and cellulase and pectate lyase and DNAse and oxidoreductase; amylase and cellulase and pectate lyase and perhydrolase and oxidoreductase; amylase and cellulase and mannanase and DNAse and perhydrolase; amylase and cellulase and mannanase and DNAse and oxidoreductase; amylase and cellulase and mannanase and perhydrolase and oxidoreductase; amylase and cellulase and DNAse and perhydrolase and oxidoreductase; amylase and pectate lyase and mannanase and DNAse and perhydrolase; amylase and pectate lyase and mannanase and DNAse and oxidoreductase; amylase and pectate lyase and mannanase and perhydrolase and oxidoreductase; amylase and pectate lyase and DNAse and perhydrolase and oxidoreductase; amylase and mannanase and DNAse and perhydrolase and oxidoreductase; lipase/cutinase and cellulase and pectate lyase and mannanase and DNAse; lipase/cutinase and cellulase and pectate lyase and mannanase and perhydrolase; lipase/cutinase and cellulase and pectate lyase and mannanase and oxidoreductase; lipase/cutinase and cellulase and pectate lyase and DNAse and perhydrolase; lipase/cutinase and cellulase and pectate lyase and DNAse and oxidoreductase; lipase/cutinase and cellulase and pectate lyase and perhydrolase and oxidoreductase; lipase/cutinase and cellulase and mannanase and DNAse and perhydrolase; lipase/cutinase and cellulase and mannanase and DNAse and oxidoreductase; lipase/cutinase and cellulase and mannanase and perhydrolase and oxidoreductase; lipase/cutinase and cellulase and DNAse and perhydrolase and oxidoreductase; lipase/cutinase and pectate lyase and mannanase and DNAse and perhydrolase; lipase/cutinase and pectate lyase and mannanase and DNAse and oxidoreductase; lipase/cutinase and pectate lyase and mannanase and perhydrolase and oxidoreductase; lipase/cutinase and pectate lyase and DNAse and perhydrolase and oxidoreductase; lipase/cutinase and mannanase and DNAse and perhydrolase and oxidoreductase; cellulase and pectate lyase and mannanase and DNAse and perhydrolase; cellulase and pectate lyase and mannanase and DNAse and oxidoreductase; cellulase and pectate lyase and mannanase and perhydrolase and oxidoreductase; cellulase and pectate lyase and DNAse and perhydrolase and oxidoreductase; cellulase and mannanase and DNAse and perhydrolase and oxidoreductase; pectate lyase and mannanase and DNAse and perhydrolase and oxidoreductase; amylase and lipase/cutinase and cellulase and pectate lyase and mannanase and DNAse; amylase and lipase/cutinase and cellulase and pectate lyase and mannanase and perhydrolase; amylase and lipase/cutinase and cellulase and pectate lyase and mannanase and oxidoreductase; amylase and lipase/cutinase and cellulase and pectate lyase and DNAse and perhydrolase; amylase and lipase/cutinase and cellulase and pectate lyase and DNAse and oxidoreductase; amylase and lipase/cutinase and cellulase and pectate lyase and perhydrolase and oxidoreductase; amylase and lipase/cutinase and cellulase and mannanase and DNAse and perhydrolase; amylase and lipase/cutinase and cellulase and mannanase and DNAse and oxidoreductase; amylase and lipase/cutinase and cellulase and mannanase and perhydrolase and oxidoreductase; amylase and lipase/cutinase and cellulase and DNAse and perhydrolase and oxidoreductase; amylase and lipase/cutinase and pectate lyase and mannanase and DNAse and perhydrolase; amylase and lipase/cutinase and pectate lyase and mannanase and DNAse and oxidoreductase; amylase and lipase/cutinase and pectate lyase and mannanase and perhydrolase and oxidoreductase; amylase and lipase/cutinase and pectate lyase and DNAse and perhydrolase and oxidoreductase; amylase and lipase/cutinase and mannanase and DNAse and perhydrolase and oxidoreductase; amylase and cellulase and pectate lyase and mannanase and DNAse and perhydrolase; amylase and cellulase and pectate lyase and mannanase and DNAse and oxidoreductase; amylase and cellulase and pectate lyase and mannanase and perhydrolase and oxidoreductase; amylase and cellulase and pectate lyase and DNAse and perhydrolase and oxidoreductase; amylase and cellulase and mannanase and DNAse and perhydrolase and oxidoreductase; amylase and pectate lyase and mannanase and DNAse and perhydrolase and oxidoreductase; lipase/cutinase and cellulase and pectate lyase and mannanase and DNAse and perhydrolase; lipase/cutinase and cellulase and pectate lyase and mannanase and DNAse and oxidoreductase; lipase/cutinase and cellulase and pectate lyase and mannanase and perhydrolase and oxidoreductase; lipase/cutinase and cellulase and pectate lyase and DNAse and perhydrolase and oxidoreductase; lipase/cutinase and cellulase and mannanase and DNAse and perhydrolase and oxidoreductase; lipase/cutinase and pectate lyase and mannanase and DNAse and perhydrolase and oxidoreductase; cellulase and pectate lyase and mannanase and DNAse and perhydrolase and oxidoreductase; amylase and lipase/cutinase and cellulase and pectate lyase and mannanase and DNAse and perhydrolase; amylase and lipase/cutinase and cellulase and pectate lyase and mannanase and DNAse and oxidoreductase; amylase and lipase/cutinase and cellulase and pectate lyase and mannanase and perhydrolase and oxidoreductase; amylase and lipase/cutinase and cellulase and pectate lyase and DNAse and perhydrolase and oxidoreductase; amylase and lipase/cutinase and cellulase and mannanase and DNAse and perhydrolase and oxidoreductase; amylase and lipase/cutinase and pectate lyase and mannanase and DNAse and perhydrolase and oxidoreductase; amylase and cellulase and pectate lyase and mannanase and DNAse and perhydrolase and oxidoreductase; lipase/cutinase and cellulase and pectate lyase and mannanase and DNAse and perhydrolase and oxidoreductase; and amylase and lipase/cutinase and cellulase and pectate lyase and mannanase and DNAse and perhydrolase and oxidoreductase.

In an embodiment, the liquid detergent composition further contains a surfactant and/or a detergent builder.

In an embodiment, the subtilisin inhibitor is a peptide aldehyde, or a hydrosulfite adduct thereof; or a phenyl boronic acid, or a derivative thereof. Preferably, the subtilisin inhibitor is an aldehyde or ketone having the formula P-(A)_{y}-L-(B)ₓ—B⁰—R* or a hydrosulfite adduct of such aldehyde, wherein:

a) R* is H (hydrogen), CH₃, CX₃, CHX₂, or CH₂X;
b) X is a halogen atom;
c) B⁰ is a single amino acid residue with L- or D-configuration of the formula —NH—CH(R)—C(═O)—;
d) x is 1, 2 or 3;
e) Bₓ is independently a single amino acid residue, each connected to the next B or to B⁰ via its C-terminal;
f) L is absent or independently a linker group of the formula —C(═O)—, —C(═O)—C(═O)—, —C(═S)—, —C(═S)—C(═S)— or —C(═S)—C(═O)—;
g) A is absent if L is absent or is independently a single amino acid residue connected to L via the N-terminal of the amino acid;
h) P is selected from the group consisting of hydrogen or if L is absent an N-terminal protection group;
i) y is 0, 1, or 2,
j) R is independently selected from the group consisting of C₁₋₆ alkyl, C₆₋₁₀ aryl or C₇₋₁₀ arylalkyl optionally substituted with one or more, identical or different, substituent's R′;
k) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂; and
l) R″ is a C₁₋₆ alkyl group.
m) x may be 1, 2 or 3.

Preferably, the inhibitor is an aldehyde having the formula P—B²—B¹—B⁰—H or an adduct having the formula P—B²—B¹—N(H)—CHR—CHOH—SO₃M, wherein

a) H is hydrogen;
b) B⁰ is a single amino acid residue with L- or D-configuration of the formula —NH—CH(R)—C(═O)—;
c) B¹ and B² are independently single amino acid residues;
d) R is independently selected from the group consisting of C₁₋₆ alkyl, C₆₋₁₀ aryl or C₇₋₁₀ arylalkyl optionally substituted with one or more, identical or different, substituent's R′;
e) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂;
f) R″ is a C₁₋₆ alkyl group; and
g) P is an N-terminal protection group.

In an embodiment, R is such that B⁰=—NH—CH(R)—C(═O)— is Phe, Tyr or Leu.

In an embodiment, B¹ is Ala, Gly or Val.

In an embodiment, B² is Arg, Phe, Tyr or Trp.

In an embodiment, x=2, L is absent, A is absent, and P is p-methoxycarbonyl (Moc) or benzyloxycarbonyl (Cbz).

In an embodiment, the inhibitor of the composition is Cbz-Arg-Ala-Tyr-H, Ac-Gly-Ala-Tyr-H, Cbz-Gly-Ala-Tyr-H, Cbz-Gly-Ala-Tyr-CF₃, Cbz-Gly-Ala-Leu-H, Cbz-Val-Ala-Leu-H, Cbz-Val-Ala-Leu-CF₃, Moc-Val-Ala-Leu-CF₃, Cbz-Gly-Ala-Phe-H, Cbz-Gly-Ala-Phe-CF₃, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO₂-Phe-Gly-Ala-Leu-H, MeSO₂-Val-Ala-Leu-H, PhCH₂O—P(OH)(O)-Val-Ala-Leu-H, EtSO₂-Phe-Gly-Ala-Leu-H, PhCH₂SO₂-Val-Ala-Leu-H, PhCH₂O—P(OH)(O)-Leu-Ala-Leu-H, PhCH₂O—P(OH)(O)-Phe-Ala-Leu-H, or MeO—P(OH)(O)-Leu-Gly-Ala-Leu-H or a hydrosulfite adduct of any of these, wherein Cbz is benzyloxycarbonyl and Moc is methoxycarbonyl. Preferably, the inhibitor is Cbz-Gly-Ala-Tyr-H or Moc-Val-Ala-Leu-H, or a hydrosulfite adduct thereof, wherein Cbz is benzyloxycarbonyl and Moc is methoxycarbonyl. Most preferably, the inhibitor is Cbz-Gly-Ala-Tyr-H, or a hydrosulfite adduct thereof, wherein Cbz is benzyloxycarbonyl.

In another aspect, the invention provides liquid detergent compositions obtainable by the methods of the invention.

The invention also provides for use of the compositions and methods above for improving the storage stability of the non-subtilisin enzyme.

The pH of the liquid detergent composition may be in the range 6.0-11; particularly in the range 6.0-10; particularly between 6.5-9.5; or between 7-9. pH may be measured directly in the composition or in a 5% solution in water.

### EXAMPLES

Chemicals used as buffers and substrates were commercial products of at least reagent grade.

### Example 1

Various peptide aldehydes were produced by a custom peptide synthesis company, all with a purity>80%. The peptide aldehydes were dissolved in DMSO to a concentration of 10 mg/mL before use.

A model liquid detergent (detergent M) was prepared for testing the protease inhibitors, as shown in Tables 1 and 2.

TABLE 1
Detergent base.

| Ingredient | % w/w |
|---|---|
| Sodium alkylethoxy sulphate (C9-15, 2EO) | 6.0 |
| Sodium dodecyl benzene sulphonate | 3.0 |
| Sodium toluene sulphonate | 3.0 |
| Oleic acid | 2.0 |
| Primary alcohol ethoxylate (C12-15, 7EO) | 3.0 |
| Primary alcohol ethoxylate (C12-15, 3EO) | 2.5 |
| Ethanol | 0.5 |
| Monopropylene glycol | 2.0 |
| Tri-sodium citrate 2H₂O | 4.0 |
| Triethanolamine | 0.4 |
| De-ionized water | Ad 100% |
| pH adjusted to 8.5 with NaOH | |

TABLE 2
Detergent M

| Ingredient | % w/w |
|---|---|
| Detergent base | 99.0 |
| Protease (specific one listed below) | 0.5 |
| Lipase (Lipex 100L, Novozymes) | 0.5 |

Further the following detergents with protease inhibitors were prepared. The detergents were placed in closed glasses at 40° C. Residual activity of protease was measured (by comparison to a reference stored at −18° C.) after 1 week, using standard enzyme analytical methods (protease measured by hydrolysis of N,N-dimethylcasein at 40° C., pH 8.3). The inhibitor levels tested relative to protease were in the range 0.5-500 molar ratio (inhibitor:protease). The data points were connected and the inhibitor dose 1 and dose 2 were determined by interpolation so that ((residual protease activity at dose 2)−(residual protease activity at dose 1))/((residual protease activity at dose 1)−(residual protease activity at no inhibitor))=0.25; and dose 2=2×dose 1.

TABLE 3

| | | | | | | ((residual activity |
|---|---|---|---|---|---|---|
| | | | | | | at dose 2) − |
| | | | | | | (residual activity |
| | | | | | | at dose 1))/ |
| | Residual | | | | | ((residual activity |
| | activity | Inhibitor | Residual | Inhibitor | Residual | at dose 1) − |
| | no | dose 1 | activity | dose 2 | activity | (residual activity |
| | inhibitor | (mg/mL) | at dose 1 | (mg/mL) | at dose 2 | at no inhibitor)) |
| Savinase and 4-FPBA | 12% | 91 | 63% | 182 | 75% | 25% |
| Savinase and Z-GAL-H | 12% | 5.7 | 50% | 11.5 | 60% | 25% |
| Savinase and Z-GAF-H | 12% | 18.7 | 75% | 37.5 | 90% | 25% |
| Savinase and Z-GAY-H | 12% | 6.2 | 73% | 12.3 | 87% | 25% |
| Savinase and Z-GA- | 12% | 8.1 | | 16.1 | | 25% |
| NHCH(CH2C6H4OH)CH(OH)SO3Na | | | | | | |
| PFP and Z-GAY-H | 15% | 25.9 | 51% | 51.9 | 60% | 25% |
| Liquanase and Z-GAY-H | 6% | 5.7 | 55% | 11.5 | 67% | 25% |
| Savinase and Z-GAy-H | 12% | 6.2 | 67% | 12.3 | 80% | 25% |

### Example 2

A commercial detergent (boric acid free, EU mid tier detergent) with added enzymes was prepared as shown in Table 4.

TABLE 4
Detergent B.

| Ingredient | % w/w |
|---|---|
| Detergent base | 97.8 |
| Protease (Savinase 16L) | 0.8 |
| Lipase (Lipex 100L) | 0.2 |
| Amylase (Termamyl 300L) | 0.6 |
| Cellulase (Novozym 342) | 0.6 |

Further the following detergents with protease inhibitors were prepared. The detergents were placed in closed glasses at 30° C. Residual activity of lipase and protease was measured (by comparison to a reference stored at −18° C.) after 4 weeks, using standard enzyme analytical methods (protease measured by hydrolysis of N,N-dimethylcasein at 40° C., pH 8.3 and lipase measured by hydrolysis of p-nitrophenyl valerate at 40° C., pH 7.7).
Z-GA-NHCH(CH2C6H4OH)CH(OH)SO3Na was added in a concentration of 12.9 mg/L (dose 1) and 26.0 mg/L (dose 2).

TABLE 5

| | No | No | Inhibitor | Inhibitor | Inhibitor | Inhibitor |
|---|---|---|---|---|---|---|
| | inhibitor | inhibitor | dose 1 | dose 1 | dose 2 | dose 2 |
| | (residual | (residual | (residual | (residual | (residual | (residual |
| | protease | lipase | protease | lipase | protease | lipase |
| | activity) | activity) | activity) | activity) | activity) | activity) |
| Savinase and Z-GA- | 21% | 3% | 85% | 10% | 95% | 40% |
| NHCH(CH2C6H4OH)CH(OH)SO3Na | | | | | | |
| %-points per dose | | | | | 10% | 30% |

### Example 3

A commercial detergent (boric acid free, EU mid tier detergent) with added enzymes was prepared as shown in Table 6.

TABLE 6
Detergent C.

| Ingredient | % w/w |
|---|---|
| Detergent base | 99.0 |
| Protease (Liquanase or Savinase) | 0.5 |
| Lipase (Lipex 100L) | 0.5 |

Further the following detergents with protease inhibitors were prepared. The detergents were placed in closed glasses at 30° C. Residual activity of lipase and protease was measured (by comparison to a reference stored at −18° C.) after 4 weeks, using standard enzyme analytical methods (protease measured by hydrolysis of N,N-dimethylcasein at 40° C., pH 8.3 and lipase measured by hydrolysis of p-nitrophenyl valerate at 40° C., pH 7.7).
Z-GA-NHCH(CH2C6H4OH)CH(OH)SO3Na was added in a concentration of 8.1 mg/L (dose 1) and 16.1 mg/L (dose 2). 4-FPBA was added in a concentration of 80 mg/L (dose 1) and 160 mg/L (dose 2).

TABLE 7

| | No | No | Inhibitor | Inhibitor | Inhibitor | Inhibitor |
|---|---|---|---|---|---|---|
| | inhibitor | inhibitor | dose 1 | dose 1 | dose 2 | dose 2 |
| | (residual | (residual | (residual | (residual | (residual | (residual |
| | protease | lipase | protease | lipase | protease | lipase |
| | activity) | activity) | activity) | activity) | activity) | activity) |
| Liquanase and Z-GA- | 11% | 0% | 89% | 50% | 89% | 75% |
| NHCH(CH2C6H4OH)CH(OH)SO3Na | | | | | | |
| %-points per dose | | | | | 0% | 25% |
| Savinase and Z-GA- | 66% | 2% | 83% | 54% | 92% | 75% |
| NHCH(CH2C6H4OH)CH(OH)SO3Na | | | | | | |
| %-points per dose | | | | | 9% | 21% |
| Savinase and 4-FPBA | 66% | 2% | 98% | 15% | 98% | 31% |
| %-points per dose | | | | | 0% | 16% |

1. A method for improving the storage stability of a non-subtilisin enzyme in a liquid detergent composition containing a subtilisin and a non-subtilisin, comprising preparing a liquid detergent composition containing a subtilisin, a non-subtilisin, and more than a concentration C of a subtilisin inhibitor; wherein C is determined as the concentration of inhibitor where the relative residual activity of the subtilisin is increased by 25% of the increase from no inhibitor to C, when the concentration of the subtilisin inhibitor is increased from C to two times C, after incubation for one week at 40° C. in Detergent M.

2. The method of claim 1, wherein the relative storage stability improvement of the non-subtilisin is higher than the relative storage stability improvement of the subtilisin.

3. The method of claim 1, wherein the liquid detergent composition is prepared by combining a subtilisin composition and a non-subtilisin enzyme composition, wherein the non-subtilisin enzyme composition comprises part of, or all of, the subtilisin inhibitor.

4. The method of claim 3, wherein the non-subtilisin enzyme composition comprises at least 50% of the subtilisin inhibitor.

5. The method of claim 1, wherein the non-subtilisin enzyme is one or more enzymes selected from the group consisting of amylase, lipase, cellulase, pectinase, mannanase, DNAse, perhydrolase, and oxidoreductase.

6. The method of claim 1, wherein the liquid composition further contains a surfactant and/or a detergent builder.

7. The method of claim 1, wherein the subtilisin inhibitor is a peptide aldehyde, or a hydrosulfite adduct thereof; or a phenyl boronic acid, or a derivative thereof.

8. The method of claim 1, wherein the inhibitor is a peptide aldehyde or ketone having the formula P-(A)y-L-(B)x-B0-R* or a hydrosulfite adduct of such aldehyde, wherein:
a) R* is H (hydrogen), CH3, CX3, CHX2, or CH2X;
b) X is a halogen atom;
c) B0 is a single amino acid residue with L- or D-configuration of the formula —NH—CH(R)—C(═O)—;
d) x is 1, 2 or 3;
e) Bx is independently a single amino acid residue, each connected to the next B or to B0 via its C-terminal;
f) L is absent or independently a linker group of the formula —C(═O)—, —C(═O)—C(═O)—, —C(═S)—, —C(═S)—C(═S)— or —C(═S)—C(═O)—;
g) A is absent if L is absent or is independently a single amino acid residue connected to L via the N-terminal of the amino acid;
h) P is selected from the group consisting of hydrogen or if L is absent an N-terminal protection group;
i) y is 0, 1, or 2,
j) R is independently selected from the group consisting of C1-6 alkyl, C6-10 aryl or C7-10 arylalkyl optionally substituted with one or more, identical or different, substituent's R′;
k) R′ is independently selected from the group consisting of halogen, —OH, —OR″, —SH, —SR″, —NH2, —NHR″, —NR″2, —CO2H, —CONH2, —CONHR″, —CONR″2, —NHC(═N)NH2; and
l) R″ is a C1-6 alkyl group.
m) x may be 1, 2 or 3.

9. The method of claim 8, wherein the inhibitor is an aldehyde having the formula P—B2-B1-B0-H or an adduct having the formula P—B2-B1-N(H)—CHR—CHOH—SO3M, wherein
a) H is hydrogen;
b) B0 is a single amino acid residue with L- or D-configuration of the formula —NH—CH(R)—C(═O)—;
c) B1 and B2 are independently single amino acid residues;
d) R is independently selected from the group consisting of C1-6 alkyl, C6-10 aryl or C7-10 arylalkyl optionally substituted with one or more, identical or different, substituent's R′;
e) R′ is independently selected from the group consisting of halogen, —OH, —OR″, —SH, —SR″, —NH2, —NHR″, —NR″2, —CO2H, —CONH2, —CONHR″, —CONR″2, —NHC(═N)NH2;
f) R″ is a C1-6 alkyl group; and
g) P is an N-terminal protection group.

10. The method of claim 8, wherein R is such that B0=—NH—CH(R)—C(═O)— is Phe, Tyr or Leu; and B1 is Ala, Gly or Val; and B2 is Arg, Phe, Tyr or Trp.

11. The method of claim 8, wherein x=2, L is absent, A is absent, and P is p-methoxycarbonyl (Moc) or benzyloxycarbonyl (Cbz).

12. The method of claim 8, wherein the inhibitor is Cbz-Arg-Ala-Tyr-H, Ac-Gly-Ala-Tyr-H, Cbz-Gly-Ala-Tyr-H, Cbz-Gly-Ala-Tyr-CF3, Cbz-Gly-Ala-Leu-H, Cbz-Val-Ala-Leu-H, Cbz-Val-Ala-Leu-CF3, Moc-Val-Ala-Leu-CF3, Cbz-Gly-Ala-Phe-H, Cbz-Gly-Ala-Phe-CF3, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO2-Phe-Gly-Ala-Leu-H, MeSO2-Val-Ala-Leu-H, PhCH2O—P(OH)(O)-Val-Ala-Leu-H, EtSO2-Phe-Gly-Ala-Leu-H, PhCH2SO2-Val-Ala-Leu-H, PhCH2O—P(OH)(O)-Leu-Ala-Leu-H, PhCH2O—P(OH)(O)-Phe-Ala-Leu-H, or MeO—P(OH)(O)-Leu-Gly-Ala-Leu-H or a hydrosulfite adduct of any of these, wherein Cbz is benzyloxycarbonyl and Moc is methoxycarbonyl.

13. The method of claim 8, wherein the inhibitor is Cbz-Gly-Ala-Tyr-H or Moc-Val-Ala-Leu-H, or a hydrosulfite adduct thereof, wherein Cbz is benzyloxycarbonyl and Moc is methoxycarbonyl.

14. A liquid detergent composition obtainable by the method of claim 1.

15. A liquid composition for use in the method of claim 3, comprising one or more non-subtilisin enzyme selected from the group consisting of amylase, lipase, cellulase, pectinase, mannanase, DNAse, perhydrolase, and oxidoreductase; and a subtilisin inhibitor which is a peptide aldehyde, or a hydrosulfite adduct thereof.


SEQUENCE LISTING (11 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 275 aa, Artificial
  BPN′ variant
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ

SEQ ID NO: 3 - protein, 4 aa, Artificial
  Subtilisin inhibitor
LGAY

SEQ ID NO: 4 - protein, 4 aa, Artificial
  Subtilisin inhibitor
FGAY

SEQ ID NO: 5 - protein, 4 aa, Artificial
  Subtilisin inhibitor
YGAY

SEQ ID NO: 6 - protein, 4 aa, Artificial
  Subtilisin inhibitor
FGAL

SEQ ID NO: 7 - protein, 4 aa, Artificial
  Subtilisin inhibitor
FGAF

SEQ ID NO: 8 - protein, 4 aa, Artificial
  Subtilisin inhibitor
FGVY

SEQ ID NO: 9 - protein, 4 aa, Artificial
  Subtilisin inhibitor
FGAM

SEQ ID NO: 10 - protein, 4 aa, Artificial
  Subtilisin inhibitor
WLVY

SEQ ID NO: 11 - protein, 4 aa, Artificial
  Subtilisin inhibitor
LGAL